(19) United States
(12) Plant Patent Application Publication
(10) Pub. No.: US 2003/0056266 P1
(43) Pub. Date: Mar. 20, 2003

(54) CHRYSANTHEMUM PLANT NAMED 'FESTIVAL TIME IMPROVED'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,494

(22) Filed: Sep. 19, 2001

(51) Int. Cl. A01H 5/00

(52) U.S. Cl. PLT/291

(57) ABSTRACT

A new variety of Chrysanthemum plant named ‘Festival Time Improved,’ having an upright free branching habit with medium vigour and slightly domed reflexing pink/dark pink bi-coloured flowers.

### LATIN NAME OF THE GENUS AND SPECIES

Botanical classification: Dendranthema grandiflora.

### VARIETY DENOMINATION

The new Chrysanthemum variety denomination is ‘Festival Time Improved.’

### BACKGROUND OF THE INVENTION

The present invention comprises a new and distinct cultivar of Chrysanthemum botanically known as Dendranthema grandiflora, and referred to by the cultivar name ‘Festival Time Improved’.

Festival Time Improved’, identified as 20278-9 was discovered as a natural mutation on a whole plant in a controlled cultivated planting of the variety ‘Festival Time.’ The new variety ‘Festival Time Improved’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Festival Time Improved’ is a pot type of Chrysanthemum plant variety having pale pink to purple decorative flowers with slightly reflexing form, giving a slightly domed capitulum.

Comparison with Parent

Plants of the new Chrysanthemum variety ‘Festival Time Improved’ are similar to the parent cultivar ‘Festival Time’ in plant habitat and growth rate. In side-by-side comparisons in Chichester, West Sussex, United Kingdom, under commercial practice, plants of the new Chrysanthemum variety ‘Festival Time Improved’ differed from plants of the parent cultivar ‘Festival Time’ in the following characteristics.

1. The new variety ‘Festival Time Improved’ produces pale pink to purple decorative colour flowers whereas the parent cultivar ‘Festival Time’ produces pale pink to lighter purple decorative colour flowers.

2. Plants of the new variety ‘Festival Time Improved’ have similar inflorescence plants as that of the parent cultivar ‘Festival Time.’

3. ‘Festival Time Improved’ has flowers that are of a more uniform habit than ‘Festival Time.’

4. ‘Festival Time Improved’ has flowers that are of a more defined colour contrast than ‘Festival Time.’

### Comparison with Other Varieties

Plants of the new Chrysanthemum variety ‘Festival Time Improved’ are dissimilar to the cultivar ‘Spring Delano’ in plant habitat and growth rate.

However, in side-by-side comparisons in Chichester, West Sussex, United Kingdom, under commercial practice, plants of the new Chrysanthemum variety ‘Festival Time Improved’ compared to plants of the cultivar ‘Spring Delano’ in the following characteristics.

1. The new variety ‘Festival Time Improved’ produces better colour flowers, whereas the cultivar ‘Spring Delano’ produces weaker colour flowers.

2. Plants of the new variety ‘Festival Time Improved’ have smaller and shorter inflorescence than plants of the cultivar ‘Spring Delano.’

3. The new variety ‘Festival Time Improved’ has a better branching habit with more flowers, compared to the variety ‘Spring Delano.’

### BRIEF DESCRIPTION OF ILLUSTRATIONS

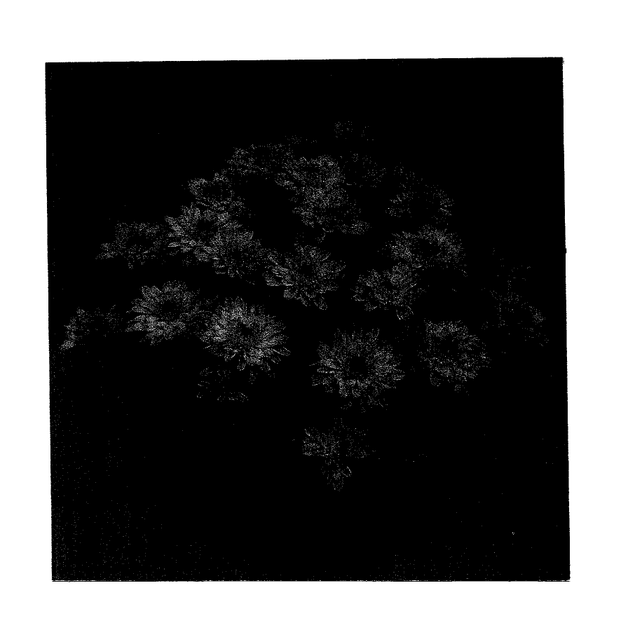

Typical specimens of the plant and flowers for the new Chrysanthemum variety ‘Festival Time Improved’ are shown in the accompanying photograph. The colours shown are as true as possible within the usual limits of this kind of illustration. FIG. 1 is a whole plant view of the new variety ‘Festival Time Improved’ grown in a pot. The plant shown in the illustration is 60 days from start of Short Days.

### DETAILED BOTANICAL DESCRIPTION

The following description of the new Chrysanthemum variety ‘Festival Time Improved’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as two B9 treatments at about 2000 ppm. To produce a commercial product the plants may be pinched once with the centre bud removed. The typical container size for commercial growth is 14 cm. It has been observed that the shelf life of the new variety is about 22 days with a response time of about 8 weeks. The new variety is suitable for growth in a temperature range of 16° C. to 25° C.

The new variety may be produced as a spray or disbud. The following description is with respect to a plant produced as Spray pot Chrysanthemums. In the description of this new Chrysanthemum variety, colour values have been taken from the Royal Horticultural Society Colour Chart (R.H.S.C.C.).

PLANT

Plant type.—Pot

Habit.—Upright and branching

Height.—Medium 18 to 20 cm

Width.—Near 27 cm

Branching characteristics.—Upright and spreading

Length of Lateral Branches.—10 to 15 cm

Number of Breaks from Pinch.—4 to 6

Stem Colour.—147B

Response Time.—54 days

Vigour.—Medium

Shelf Life.—Near 22 days

Disease.—No observations

Pest.—No Observations

Growth retardant type and treatment.—B9 85% 2 applications of 2 gram/litre at 2 days, 21 days after propagating unrooted vegetative cuttings. The plants were grown for two weeks in Long Day conditions (20 hours of light) and then transferred to Short Day conditions (13 hours of dark).

Propagation.—Type: Vegetative propagation via stem cuttings Time to rooting: 12-14 days with soil temperatures of 18° C. Rooting habit: Fine and fibrous

FOLIAGE

Number of Leaves per Lateral Branch.—8 to 11

Compound or single.—Single

Arrangement of Leaves.—Alternate Shape of Leaf: Typically 5 lobed, ovate Size of Leaf: Width (cm): Near 4 cm Length (cm): Near 7 cm Leaf Apex: Acute/Rounded Base: Obtuse Attachment: Petioled Aspect: Slightly undulating Margin: Palmately lobed Surface characteristics: Top: Pubescent Bottom: Pubescent

Petiole.—Colour: Near 137B Length: 0.8 to lcm

Venation.—Net, prominent midvein and lateral veins at under side. Colour: upper side: Near 147B under side: Near 147B

Colour.—Mature Leaf, upper side: Near 137A; under side: Near 137C Young Leaf, upper side: Near 137A; under side: Near 137C

FLOWER

Flower Appearance.—Slightly domed and reflexing. Circular

Flower type.—Decorative

Flower Form.—Slightly domed

Flower shape.—Circular

Flower Habit.—Upright

Number of blossoms per branch.—3 to 5

Inflorescence form.—Cyme Depth of fully expanded blossoms: Near 2½ cm Diameter of fully expanded blossoms: Near 7 cm

Sepals.—Number: 21 to 30 Colour: Near 137A Length: 0.6 to 1.1 cm Width: Near 0.3 cm Texture/Appearance: Pubescent Peduncles: Peduncle length is shortest at the terminal flower and progressively longer moving down the stem; longest length is on average 10 cm; shortest length on average 5 cm. Length: 6 cm Colour: Near 137B Surface: Pubescent Habit: Slightly curving Strength: Medium

Pedicel.—Length: Terminal: Near 4 cm; Lateral: Near 6 cm Colour: Near 137B Surface: Pubescent Habit: Slightly curving Strength: Medium

Ray Florets.—Form/Shape: Reflexing Texture/Appearance: Shiny Number per flower: 200 to 220 Length: 1.2 to 2.7 cm; Width: 0.4 to 0.8 cm Apex: Slightly dentate Base: Tapered Margin: Entire

Disc florets.—No known Disc Florets

Fragrance.—Slight

Flower Bud (at onset of colour).—Length: Near 0.9 cm Diameter: Near 1.0 cm Form/shape: Globular

General flower colour.—Pale pink with darker pink centre to flower. 1. Ray florets, upper side: Immature: Near 67B Mature: Paler than 36D Older/Fading: Near 157 D 2. Ray florets, under side: Immature: Near 67B Mature: Paler than 36D Older/Fading: Near 157D 3. Disc florets: N/A 4. Bud: Near 138A Flower progression: Flowers reflex and fade slightly with age, but colour differential is maintained well into pot life.

REPRODUCTIVE ORGANS

Ray Florets.—Pistillate Pistil number: 100+Stigma colour: Near 6C s Stigma shape: Forked Style Colour: Near 6C Style Length: Near 0.5 cm Disc Florets: Pistillate: not observed Staminate: not observed

What is claimed is:

1. A new and distinct variety of Chrysanthemum plant, substantially as described and illustrated herein.
